(19) United States
(12) Patent Application Publication
(10) Pub. No.: US 2008/0287653 A1
(43) Pub. Date: Nov. 20, 2008

(54) METHOD FOR PRODUCING INHIBITORS AND INHIBITORS FORMED THEREFROM

(75) Inventors: Weiping ZHENG, Copley, OH (US); David George FATKINS, Clinton, OH (US)

Correspondence Address: ROETZEL AND ANDRESS, 222 SOUTH MAIN STREET, AKRON, OH, 44308, US

(73) Assignee: THE UNIVERSITY OF AKRON, Akron, OH (US)

(21) Appl. No.: 12/119,054

(22) Filed: May 12, 2008

Related U.S. Application Data
(60) Provisional application No. 60/928,811, filed on May 11, 2007.

(51) Int. Cl. C07K 7/08; C07K 1/00

(52) U.S. Cl. 530/326; 530/345

(57) ABSTRACT

The present invention relates to methods for producing inhibitors for protein deacetylases, and to the compounds and/or products produced by such methods. More specifically, the present invention relates to methods for producing inhibitors for human class III protein deacetylases or sirtuins, and to the compounds and/or products produced by such methods. The present invention provides the transformation of peptide substrates to potent peptide inhibitors by replacement of N^{ε}-thioacetyl-lysine for N^{ε}-acetyl-lysine.

### FIELD OF THE INVENTION

The present invention relates to methods for producing inhibitors for protein deacetylases, and to the compounds and/or products produced by such methods. More specifically, the present invention relates to methods for producing inhibitors for human class III protein deacetylases or sirtuins, and to the compounds and/or products produced by such methods.

### BACKGROUND OF THE INVENTION

Protein acetyltransferases and protein deacetylases are the two families of enzymes that respectively catalyze the specific lysine N^{ε}-acetylation and deacetylation on proteins such as the core histone proteins, various transcription factors, alpha-tubulin, and acetyl-coenzyme A synthetases that are respectively involved in gene transcriptional, cytoskeletal, and metabolic control. FIG. 1 sets forth this relationship. Based on homology with yeast transcriptional repressors, phylogeny analysis, and different cofactor requirements, human protein deacetylase enzymes have been categorized into Class I (HDAC1,2,3,8); Class II (HDAC4,5,6,7,9,10), Class III (SIRT1,2,3,4,5,6,7), and Class IV (HDAC11 and its related enzymes) subfamilies. “HDAC” is the abbreviation for protein deacetylase that is named after the first discovered protein substrate histone. Class I, II, and IV enzymes, also collectively known as classical enzymes, all require a catalytic zinc (Zn²⁺) for activity, whereas Class III enzymes, also known as sirtuins, require coenzyme nicotinamide adenine dinucleotide (NAD⁺) for activity. Among the 7 human sirtuins, only SIRT1, SIRT2, SIRT3, SIRT5, and SIRT6 have been demonstrated to be bona fide protein deacetylases. In addition, SIRT6 is also an ADP-ribosyltransferase, as is SIRT4. An enzymatic activity for SIRT7 has not been identified.

Chemical modulation, or inhibition and activation, of these enzymes offers therapeutic benefits for treating human diseases, including but not limited to metabolic and age-related diseases and cancer. In addition, this modulation provides a chemical biological approach to further deciphering the biology of these enzymes.

As compared to the long-standing active pursuit of inhibitors of Zn²⁺-dependent classical enzymes, the development of inhibitors/activators of the NAD⁺-dependent protein deacetylases has only recently been considered. Regarding the inhibitor development for these latter enzymes, only two research reports disclose the discovery of potential selective inhibitors that demonstrate not only potent but also selective activity. An indole-based SIRT1 inhibitor (IC₅₀˜98 nM) has been reported by Napper, et al., Discovery of Indoles as Potent and Selective Inhibitors of the Deacetylase SIRT1, J. Med. Chem., 48 (2005) 8045-8054, and a SIRT2 inhibitor (IC₅₀˜3.5 μM) has been reported by Outeiro, et al., Sirtuin2 Inhibitors Rescue Alpha-Synuclein-Mediated Toxicity in Models of Parkinson's Disease, Science 2007, 317, 516-519. Weak or micromolar level inhibition and/or non-selective inhibition, or the inhibiting of multiple deacetylases within the class III subfamily and/or also inhibiting enzymes outside of this subfamily hamper of all other currently reported inhibitors whose potency and selectivity have been sufficiently addressed. Therefore, developing a novel inhibition strategy and the related inhibitors for human NAD⁺-dependent protein deacetylases presents a unique problem that needs to be addressed.

### SUMMARY OF THE INVENTION

The invention relates to methods for producing inhibitors for protein deacetylases, and to the compounds and/or products produced by such methods. More specifically, the present invention relates to methods for producing inhibitors for human class III protein deacetylases or sirtuins, and to the compounds and/or products produced by such methods.

In one embodiment the invention provides a method for transforming a peptide substrate into a selective peptide inhibitor comprising: a) providing a peptide substrate containing N^{ε}-acetyl-lysine; b) providing an L-N^{α}-Fmoc-N^{ε}-thioacetyl-lysine building block; and c) reacting the building block to replace N^{ε}-acetyl-lysine in the peptide substrate with N^{ε}-thioacetyl-lysine from the building block, wherein the resulting peptide exhibits selective inhibition for enzyme activity.

In another embodiment, the invention provides a method for producing inhibitors for human class III protein deacetylases comprising: a) providing a peptide substrate containing N^{ε}-acetyl-lysine; b) providing an L-N^{α}-Fmoc-N^{ε}-thioacetyl-lysine building block; and c) reacting the building block to replace N-acetyl-lysine in the peptide substrate with N^{ε}-thioacetyl-lysine from the building block, wherein the resulting peptide exhibits selective inhibition for class III protein deacetylase enzymes.

In yet another embodiment the invention provides a peptide-based human sirtuin inhibitor comprising an N^{ε}-thioacetyl-lysine-containing peptide-based human sirtuin exhibiting resistance to classical HDAC-enzyme dethioacetylation.

These and other embodiments will become known to the reader based on the following disclosure.

### BRIEF DESCRIPTION OF THE FIGURES

FIG. 1 is an illustration of the lysine N^{ε}-acetylation and deacetylation reactions catalyzed respectively by protein acetyltransferases and protein deacetylases.

FIG. 2 is a structural comparison of N^{ε}-acetyl-lysine and N^{ε}-thioacetyl-lysine according to the invention.

FIG. 3 is the synthesis scheme for L-N-Fmoc-N^{α}-thioacetyl-lysine.

FIG. 4 is a list of peptides produced and evaluated according to the invention.

FIG. 5 is a panel of representative HPLC chromatograms from HDAC8 assays with peptides 1b, 2, and 3a.

FIG. 6 is the synthesis scheme for L-N^{α}-acetyl-N^{ε}-thioacetyl-lysine.

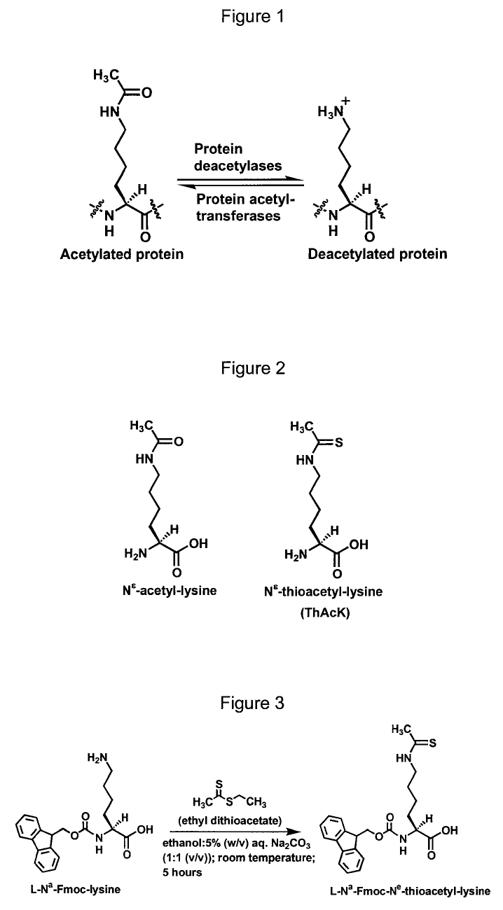

### DETAILED DESCRIPTION OF THE INVENTION

The present invention relates to methods for producing inhibitors for protein deacetylases, and to the compounds and/or products produced by such methods. More specifically, the present invention relates to methods for producing inhibitors for human class III protein deacetylases or sirtuins, and to the compounds and/or products produced by such methods. The present invention provides the transformation of peptide substrates to potent peptide inhibitors by replacement of N^{ε}-thioacetyl-lysine for N^{ε}-acetyl-lysine, the structures of which are shown in FIG. 2.

In one embodiment, the present invention relates to Compound I having the following formula:

R¹NH-Φₘ-(ThAcK)-Φₙ-COR² (I)

wherein, ThAcK is L-N^{ε}-thioacetyl-lysine; φ is one of the 20 naturally occurring L-amino acids and their D-counterparts (i.e. Alanine, Arginine, Asparagine, Aspartic acid, Cysteine, Glutamic acid, Glutamine, Glycine, Histidine, Isoleucine, Leucine, Lysine, Methionine, Phenylalanine, Proline, Serine, Threonine, Tryptophan, Tyrosine, Valine); m is 0-10; n is 0-10, and when m and/or n is >1, φ may be the same amino acid or different amino acids; R¹ is hydrogen (H), acetyl (CH₃CO), tert-butyloxycarbonyl (tBoc); R² is hydroxyl (OH), amino (NH₂).

Compound I is synthesized using the L-N^{α}-Fmoc-N^{ε}-thioacetyl-Lysine building block. The building block can be synthesized according to the synthetic scheme shown in FIG. 3. Specifically, L-N^{α}-Fmoc-N^{α}-thioacetyl-Lysine may be synthesized by the condensation of L-N^{α}-Fmoc-lysine with ethyl dithioacetate under slightly basic conditions. The inventors of the present invention have further demonstrated that L-N^{α}-Fmoc-N^{ε}-thioacetyl-Lysine building block can be used directly for the incorporation of L-N^{ε}-thioacetyl-lysine (ThAcK) into a peptide sequence, according to the Fmoc chemistry-based solid phase peptide synthesis (SPPS), Wellings, D. A., Atherton, E., Methods Enzymol. 1997, 289,44. The resulting peptides, having the formula shown above as Compound I, behave as potent inhibitors of surtuins.

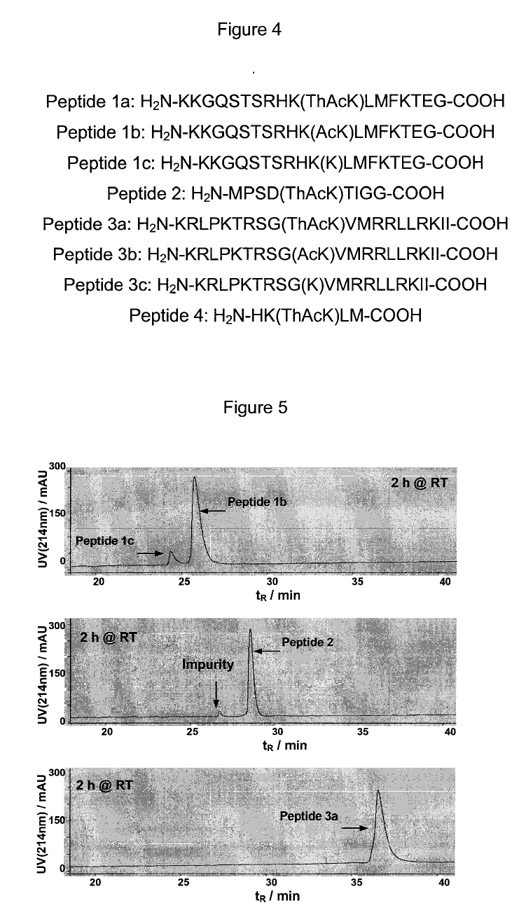

In another embodiment of the present invention, the human p53 tumor suppressor protein C-terminal peptide, corresponding to amino acid residues 372-389 containing N^{ε}-thioacetyl-lysine at the 382 position, was synthesized according to the disclosure herein. This peptide is set forth as peptide 1a in FIG. 4. When it was evaluated together with the corresponding N^{ε}-acetyl-lysine-containing and lysine-containing peptides, i.e. peptides 1b and 1c in FIG. 4, serving respectively as the substrate and the product of SIRT1-catalyzed reaction, it was found to be a potent inhibitor for SIRT1 with IC₅₀ value being 1.7±0.4 uM. As used herein, “IC₅₀” is defined as the concentration of an inhibitor to achieve 50% inhibition of an enzyme-catalyzed reaction. The data supporting the foregoing is set forth in Table 1.

In yet another embodiment of the present invention, the human α-tubulin peptide, corresponding to amino acid residues 36-44 containing N-thioacetyl-lysine at the 40 position was also synthesized. This peptide is set forth as peptide 2 in FIG. 4. When this peptide was evaluated together with the corresponding N^{ε}-acetyl-lysine-containing and lysine-containing peptides, i.e. Peptides 1b and 1c in FIG. 4, serving respectively as the substrate and the product of SIRT2-catalyzed reaction, it was also found to be a potent inhibitor for SIRT2 with IC₅₀ value being 11.4±1.1 u M. This data is also contained in Table 1.

In still another embodiment of the present invention, the human Acetyl-coenzyme A synthetase 2 peptide, or AceCS2 peptide, corresponding to amino acid residues 633-652 and containing N′-thioacetyl-lysine at the 642 position, was also synthesized. This peptide is set forth as peptide 3a in FIG. 4. When this peptide was evaluated together with the corresponding N^{ε}-acetyl-lysine-containing and lysine-containing peptides, i.e. Peptides 3b and 3c in FIG. 4, serving respectively as the substrate and the product of SIRT3-catalyzed reaction, it was also found to be a potent inhibitor for SIRT3, with IC₅₀ value being 4.5±2.0 uM. As with the above peptides, Table 1 supports this finding.

The forgoing thus demonstrated that, replacing N^{ε}-thioacetyl-lysine for N^{ε}-acetyl-lysine in a peptide substrate represented a general strategy to develop potent inhibitors of human NAD⁺-dependent protein deacetylase enzymes.

Peptides 1a, 2, and 3a were further evaluated for their possible selective inhibition among SIRT1, SIRT2, and SIRT3 surtuins. With reference to Table 1, peptide 1a was found to have a comparable inhibition potency against SIRT2 to that against SIRT1, but an inhibition potency approximately 35-fold weaker against SIRT3. Peptide 2 was found exhibit inhibition of about 10-fold weaker for SIRT1 and about 40-fold weaker SIRT3 as compared to its inhibition against SIRT2. Peptide 3a was found to be a comparably potent inhibitor for both SIRT2 and SIRT3, but to exhibit about 5-fold stronger inhibition with respect to SIRT1.

Though not wishing to be bound by any particular theory, the rationale for evaluating the relative inhibition potencies of peptides 1a, 2, and 3a among SIRT1, SIRT2, and SIRT3 was based on the determination that, if the amino acid residues surrounding N^{ε}-thioacetyl-lysine in these peptides defined different “addresses” that were to be recognized by different enzymes, selective inhibition ought to be obtained. The different degrees of selective inhibition demonstrated during the evaluation support this rationale.

Notwithstanding the foregoing, further consideration of the strong inhibition of peptide 1a against SIRT2 and peptide 3a against both SIRT1 and SIRT2, as shown in Table 1, given that peptides 1a and 3a were based on peptide templates derived from the SIRT1 physiological substrate human p53 protein and the SIRT3 physiological substrate human AceCS2, respectively, was warranted Again, while not wishing to be bound by any specific theory, it is believed that under certain in vivo conditions, SIRT2 accepts human p53 protein and AceCS2 as its substrates and S1RT1 accepts AceCS2 as its substrate. Alternatively, the in vitro experimental data with purified recombinant enzymes may not fully account for the substrate selection by these enzymes in vivo, which could also be regulated by both spatial and temporal mechanisms.

The foregoing establishes that in addition to conferring potent inhibition for human sirtuins, replacing N^{ε}-thioacetyl-lysine for N^{ε}-acetyl-lysine in a peptide substrate additionally represents a general and efficient strategy to develop selective inhibitors of human sirtuins.

The inventors of the present invention previously demonstrated that peptides 1a and 1b, as set forth in FIG. 4, were comparably de(thio)acetylated by HDAC8. Therefore, when placed within an appropriate amino acid sequence, the thioacetyl group can serve as a functional mimic for the acetyl group for the enzymatic deacetylation reaction catalyzed by HDAC8. It is also supported from the inventors' earlier work employing HeLa nuclear extracts enriched in HDAC1 and HDAC2, as well as the purified HDAC1 and HDAC2 enzyme preparations, that when N^{ε}-thioacetyl-lysine is substituted for N^{ε}-acetyl-lysine within an appropriate amino acid sequence, the thioacetyl group can serve as a functional mimic for the acetyl group only for HDAC8-catalyzed reaction, i.e. the thioacetyl group is selective for HDAC8. See Fatkins et al., A Spectrophotometric Assay for Histone Deactylase 8, Anal. Biochem 2008, 372, 82-88.

Based on the foregoing, the result was extrapolated to determine if peptides 2 and 3a could also be dethioacetylated by HDAC8, because the liability (what does this term mean here?) of a N^{ε}-thioacetyl-lysine-containing peptide toward HDAC8 is expected to diminish its value as a chemical biological research tool or a potential therapeutic agent.

To test the foregoing premise, peptides 1b, 2, and 3a were allowed to be incubated for 2 hours at room temperature in the HDAC8 assay buffer in a HPLC-based HDAC8 assay. While approximately 10% substrate conversion to product from peptide 1b was observed, no detectable formation of the dethioacetylated peptide products was observed from either of peptides 2 or 3a. FIG. 5 provides representative HPLC chromatograms from HDAC8 assays with peptides 1b, 2, and 3a. All assays were performed in duplicate and essentially the same HPLC chromatograms were obtained for the duplicates. The small peak with t_{R}˜27 minutes in the second chromatogram was from a minor impurity in the purified peptide 2 sample, rather than the dethioacetylated product. The lack of detectable dethioacetylation of peptide 3a was apparent from the absence of a peak with t_{R}˜34 minutes, shown for peptide 3c in the third chromatogram. The results indicate that, unlike peptides 1a and 1b, peptides 2 and 3a could not be dethioacetylated by HDAC8 under the same experimental conditions.

The foregoing then establishes that in addition to conferring potent inhibition for human sirtuins, replacing N^{ε}-thioacetyl-lysine for N^{ε}-acetyl-lysine in a peptide substrate additionally represents a general and efficient strategy to develop selective inhibitors of human sirtuins. Furthermore, a potent and selective N^{ε}-thioacetyl-lysine-containing peptide-based human sirtuin inhibitor showing resistance to enzymatic dethioacetylation by classical HDAC enzymes has been identified.

Even though these peptide-based inhibitors might not be cell permeable, they can be ferried inside a cell by their conjugation, e.g. via a disulfide linkage, to various types of protein transduction domain (PTD) peptides. Once a PTD peptide carries an inhibitor as cargo across cellular membranes, the cargo can be released inside a cell following the cleavage of a disulfide linkage due to a reductive intracellular environment. This is one way to introduce the current inhibitors into a cell for intracellular activity, though other introduction mechanisms may also be used.

Still further, the peptide-based inhibitors are potentially valuable lead compounds for developing inhibitors with enhanced potency, selectivity, metabolic stability, and cellular membrane permeability. For example, the peptide-based inhibitors can be used to develop in vivo applications. This may be accomplished by following a stepwise strategy consistent with the following, though other strategies known to those skilled in the art will also be useful. In the strategy proposed herein, a first step may include obtaining structurally more manageable minimal peptide sequences via peptide truncation. Further, the peptides obtained may be used to construct and screen a focused peptide library in which all the library members will have N^{ε}-thioacetyl-lysine occupying their middle positions. This may be focused on, for example, various human sirtuins. Finally, the results may be employed to perform further medicinal chemistry manipulations on library hits. While maintaining the potency and selectivity of hits, their metabolic stability and cellular membrane permeability could be potentially enhanced through the minimization of their peptidic nature.

In keeping with the above-stated strategy to develop improved inhibitors suitable for in vivo applications, a preliminary structure-activity-relationship study on peptide 1a has been performed. In this embodiment of the present invention, peptide 1a was truncated to a simpler peptide with ThAcK occupying its middle position, according to peptide 4 in FIG. 4. An IC₅₀ value of 10.4±0.3 uM, as set forth in Table 1, was obtained for this simpler peptide to inhibit SIRT1, which is a modest decrease of approximately 6-fold, in inhibition potency following the truncation.

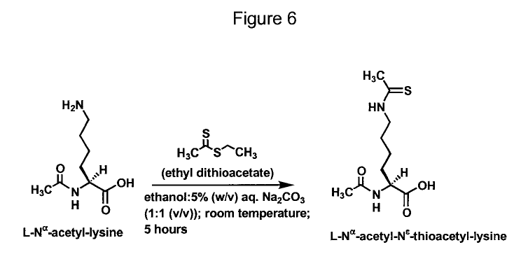

In another embodiment of the present invention, peptide 1a was truncated to L-N^{α}-acetyl-N^{ε}-thioacetyl-lysine. The resulting compound did not exhibit any SIRT1 inhibition at 2 mM, as shown in Table 1. FIG. 6 depicts the synthesis of L-N^{α}-acetyl-N-thioacetyl-lysine, which may be synthesized by the condensation of L-N^{α}-acetyl-lysine with ethyl dithioacetate under slightly basic conditions.

Based on the foregoing L-N^{α}-acetyl-N^{ε}-thioacetyl-lysine results, L-N^{α}-Fmoc-N^{ε}-thioacetyl-lysine was tested to determine the use thereof as a potential SIRT1 inhibitor. As shown in Table 1, L-N^{α}-Fmoc-N^{ε}-thioacetyl-lysine exhibited a very weak SIRT1 inhibition with an IC₂₅ value being 2,000 uM. This IC₂₅ value, as used herein, refers to the concentration of an inhibitor to achieve 25% inhibition of an enzyme-catalyzed reaction.

The forgoing results demonstrate that a peptide-based sirtuin inhibitor with greater than 5 amino acid residues could be simplified to a shortened or truncated peptide with 5 amino acid residues without a drastic loss of human sirtuin inhibition potency. However, further structural simplification by removing more amino acid residues could diminish the inhibition potency of the parent peptide-based inhibitors dramatically.

In still another embodiment, the present invention relates to a pharmaceutical preparation that contains Compound I, as defined above, for preventing or treating a condition or disorder that is mediated by the protein deacetylation reaction catalyzed by a human class III protein deacetylase, including SIRT1, SIRT2, SIRT3, SIRT5, and SIRT6. Such conditions or disorders include, but are not limited to, cancer and Parkinson's Disease. This type of pharmaceutical preparation may take the form of an intravenous or intramuscular injection, though the use of Compound I is not so limited, and may be administered by any available means. this embodiment further includes a method to provide such a pharmaceutical preparation.

In another further embodiment, the present invention provides a method of modulating the protein deacetylation activity of a human class III protein deacetylase, including SIRT1, SIRT2, SIRT3, SIRT5, and SIRT6. This method may be based on the use of Compound I, as defined hereinabove.

In still another embodiment, the present invention provides a novel inhibitor design strategy by which potent and selective L-N^{ε}-thioacetyl-lysine-containing inhibitors for human class III protein deacetylase enzymes can be obtained.

The following paragraphs set forth exemplary processing conditions and processes, which are provided to assist the reader in understanding and repeating the invention disclosed herein.

Synthesis of N^{α}-Fmoc-N^{ε}-thioacetyl-lysine. A 5% (w/v) aqueous solution of Na₂CO₃ (2.12 mL) was added dropwise, at 0° C., to a stirred suspension of N^{α}-Fmoc-lysine (368 mg, 1.0 mmol) in EtOH (2.12 mL). Ethyl dithioacetate (126 μL, 1.1 mmol) was then added dropwise at 0° C. After the addition was complete, the reaction mixture was stirred at room temperature for 5 hours before the addition of a 50% (v/v) solution of EtOH in double deionized water (ddH₂O) (3 mL). The ethanol was removed under reduced pressure and the residual aqueous solution was acidified with 6 N HCl to pH of about 1 to 2 and extracted with dichloromethane. The combined organics were washed with brine, dried over anhydrous Na₂SO₄, filtered, and concentrated under reduced pressure, generating an oily residue from which the product was isolated via silica gel column chromatography as an oily white solid (302 mg, 71%) exhibiting the following characteristics: ¹H NMR (300 MHz, CDCl₃): δ 9.33 (br, 1H, C(═S)NH), 7.97-7.30 (m, 8H, Harom), 5.73 (br d, 1H, J=6.6 Hz, OC(═O)NH), 4.50-4.05 (m, 4H, fluorenyl Hg, CH₂O, and Ha), 3.59 (br, 2H, CH₂NH), 2.49 (s, 3H, CH₃), 1.89-1.28 (m, 6H, CH₂CH₂CH₂); ¹³C NMR (75 MHz, CDCl3): δ 200.9 (C(═S)NH), 176.2 (COOH), 156.7 (NHC(═O)O), 143.5 (Cₐᵣₒₘ), 141.3 (Cₐᵣₒₘ), 128.0 (Cₐᵣₒₘ), 127.3 (Cₐᵣₒₘ), 125.1 (Cₐᵣₒₘ), 120.2 (Cₐᵣₒₘ), 67.4 (CH₂O), 53.5 (Cₐₗₚₕₐ), 47.1 (fluorenyl C₉), 46.1 (CH₂NH), 33.9 (CH₂), 32.0 (CH₂), 27.1 (CH₂), 22.8 (CH₃); HRMS (ESI) calcd for C₂₃H₂₆N₂NaO₄S ([M+Na]⁺) 449.15055; found: 449.14955.

Synthesis of N^{α}-acetyl-N^{ε}-thioacetyl-lysine. N^{ε}-acetyl-N′-thioacetyl-lysine was prepared according to the invention by following the same procedure as set forth above for the synthesis of N^{α}-Fmoc-N^{ε}-thioacetyl-lysine. An oily white solid exhibiting the following characteristics was obtained: ¹H NMR (300 MHz, DMSO-d₆): δ 10.08 (br, 1H, C(═S)NH), 7.64 (br d, 1H, J=7.2 Hz, C(═O)NH), 3.98 (br, 1H, Ha), 3.41 (br, 2H, CH₂NH), 2.36 (s, 3H, CH₃C(═S)), 1.82 (s, 3H, CH₃(C═O)), 1.66-1.27 (m, 6H, CH₂CH₂CH₂); ¹³C NMR (75 MHz, DMSO-d6): δ 198.7 (C(═S)NH), 174.9 (COOH), 168.6 (C(═O)NH), 53.3 (Ca), 45.6 (CH₂NH), 32.8 (CH₂), 32.0 (CH₂), 27.1 (CH₂), 23.0 (CH₃), 22.9 (CH₃); MS (ESI): m/z 247 [M+H]⁺.

Peptide synthesis and purification. All peptides reported herein were synthesized based on the Fmoc chemistry strategy on a commercial peptide synthesizer, such as that available from Protein Technologies Inc., Tucson, Ariz., USA. Except N^{α}-Fmoc-N^{ε}-thioacetyl-lysine, all other Fmoc-protected amino acids and pre-loaded Wang resins were purchased from Novabiochem (La Jolla, Calif., USA), N^{α}-Fmoc-N^{ε}-thioacetyl-lysine was synthesized from N^{α}-Fmoc-lysine and ethyl dithioacetate as described above. For each coupling reaction, 4 equivalents of a Fmoc-protected amino acid, 3.84.0 equivalents of the coupling reagent 2-(1H-benzotriazole-1-yl)-1,1,1,3,3-tetramethylaminium hexafluorophosphate (HBTU) and the additive N-hydroxybenzotriazole (HOBt) were used in the presence of 0.4 M 4-methylmorpholine (NMM)/DMF, and the coupling reaction was allowed to proceed at room temperature for 1 hour. A 20% (v/v) piperidine/DMF solution was used for Fmoc removal. All the peptides were cleaved from the resins by reagent K (i.e. 83.6% (v/v) trifluoroacetic acid, 5.9% (v/v) phenol, 4.2% (v/v) ddH₂O, 4.2% (v/v) thioanisole, 2.1% (v/v) ethanedithiol), precipitated in cold diethyl ether, and purified by reversed-phase HPLC on a preparative C18 column (100 Å, 2.14×25 cm). The column was eluted with a gradient of ddH₂O containing 0.05% (v/v) of trifluoroacetic acid and acetonitrile containing 0.05% (v/v) of trifluoroacetic acid at 10 mL/min and monitored at 214 nm. The pooled HPLC fractions were stripped of acetonitrile and lyophilized to give all peptides as puffy white solids. Peptide purity (>95%) was verified by reversed-phase HPLC on an analytical C18 column (100 Å, 0.46×25 cm). The column was eluted with a gradient of ddH₂O containing 0.05% (v/v) of trifluoroacetic acid and acetonitrile containing 0.05% (v/v) of trifluoroacetic acid at 1 mL/min and monitored at 214 nm. The molecular weights of all purified peptides were confirmed by matrix assisted laser desorption ionization-time of flight (MALDI-TOF) or electrospray ionization (ESI) mass spectrometric analysis. The following details the results of this analysis. Peptide 1a: MS (MALDI-TOF) m/e 2149 [M+H]⁺; Peptide 1b: MS (MALDI-TOF) m/e 2133 [M+H]⁺; Peptide 1c: MS (MALDI-TOF) m/e 2091 [M+H]⁺; Peptide 2: MS (MALDI-TOF) m/e 964 [M+H]⁺; Peptide 3a: MS (MALDI-TOF) m/e 2508 [M+H]⁺; Peptide 3b: MS (MALDI-TOF) m/e 2492 [M+H]⁺; Peptide 3c: MS (MALDI-TOF) m/e 2450 [M+H]⁺; and Peptide 4: MS (ESI) 714 [M+H]⁺.

Inhibition assays with purified SIRT1, SIRT2, and SIRT3. GST-SIRT1 that could be produced in a laboratory was used for the SIRT1 inhibition assay. SIRT2 and SIRT3 are obtainable from commercial sources known to those skilled in the art. Consistent HPLC-based assay procedures were used for the inhibition assays with SIRT1, SIRT2, and SIRT3. In brief, an inhibition assay solution had the following components: 25 mM (or 50 mM for SIRT2 assay) Tris.HCl (pH 8.0) 137 mM NaCl, 2.7 mM KCl, 1 mM MgCl₂, 1 mg/mL BSA (SIGMA Cat. #A3803 with reduced fatty acid content, for SIRT2 assay only), 0.5 mM β-NAD⁺, 0.3 mM peptide substrate (peptide 1b for SIRT1 and SIRT2 assays; peptide 3b for SIRT3 assay), an inhibitor (peptide 1a, 2, or 3a) with varied concentrations including 0, and an enzyme (GST-SIRT1, 0.15 μM; SIRT2, 0.3 μM; or SIRT3, 2.0 μM). FIG. 4 shows the sequences for these peptides. An enzymatic reaction was initiated by the addition of an enzyme at 37° C. and was allowed to be incubated at 37° C.: 10 minutes as follows: for SIRT1 assay, or 60 minutes for SIRT2 and SIRT3 assays. Incubation was quenched at the indicated time with the following stop solution: 100 mM HCl and 0.16 M acetic acid. The conversion of the limiting substrate to the product was maintained at ≦12%. The quenched assay solutions were directly injected into a reversed-phase HPLC C18 column (100 Å, 0.46×25 cm), eluting with the following gradients of ddH₂O containing 0.05% (v/v) trifluoroacetic acid (mobile phase A) and acetonitrile containing 0.05% (v/v) trifluoroacetic acid (mobile phase B): linear increase from 0% B to 35% B (for SIRT1 assay) or 40% B (for SIRT2 and SIRT3 assays) from 0-40 minutes (1 mL/min), and UV monitoring at 214 nm. The enzymatic deacetylation products (peptide 1c in SIRT1 and SIRT2 assays; peptide 3c in SIRT3 assay) were confirmed by their comigration with the chemically synthesized authentic samples and by MALDI-TOF mass spectrometric analysis, and were quantified by HPLC peak integration and comparison with those of synthetic authentic samples. Under the same assay conditions, no detectable formation of the deacetylation products (i.e. peptides 1c and 3c) was observed for non-enzymatic reactions. Furthermore, under the same assay conditions, peptides 1a and 3a did not give rise to detectable formation of peptides 1c and 3c, respectively, via dethioacetylation, though detectable formation of the corresponding dethioacetylated peptide from peptide 2 was observed under the same assay conditions. All peptide stock solutions were prepared in ddH₂O. IC₅₀ values were estimated from Dixon plots (1/v₀ vs. [inhibitor]) as an indication of the inhibition potency.

Assay with purified HDAC8. The purified HDAC8 could be obtained from a commercial source for use with a HPLC-based HDAC8 assay. In brief, a HDAC8 assay solution had the following components: 25 mM Tris.HCl (pH 8.0), 137 mM NaCl, 2.7 mM KCl, 1 mM MgCl₂, 1 mg/mL BSA (SIGMA Cat. #A3803 with reduced fatty acid content), 0.3 mM peptide 1b, 2, or 3a, and 1.5 μM HDAC8. FIG. 4 shows the sequences for these peptides. An enzymatic reaction was initiated by the addition of HDAC8 at room temperature and was allowed to be incubated at room temperature for 2 hours before being quenched with the following stop solution: 1.0 M HCl and 0.16 M acetic acid. The quenched assay solutions were directly injected into a reversed-phase HPLC C18 column (100 Å, 0.46×25 cm), eluting with the following gradients of ddH₂O containing 0.05% (v/v) trifluoroacetic acid (mobile phase A) and acetonitrile containing 0.05% (v/v) trifluoroacetic acid (mobile phase B): linear increase from 0% B to 35% B (for the assay with peptide 2 or 1b) or 40% B (for the assay with peptide 3a or 1b) from 0-40 minutes (1 mL/min), and UV monitoring at 214 nm. Under the HDAC8 assay conditions, the deacetylated peptide (i.e. peptide 1c) was formed from peptide 1b, as shown in FIG. 5, however, no detectable formation of the corresponding dethioacetylated peptides from peptides 2 and 3a was observed. The enzymatically formed peptide 1c was confirmed by its comigration with the chemically synthesized authentic sample and by MALDI-TOF mass spectrometric analysis, and was quantified by HPLC peak integration and comparison with that of synthetic authentic sample. Under the same assay conditions, no detectable formation of peptide 1c was observed for non-enzymatic reactions.

TABLE 1
Human Sirtuin Inhibitor Evaluation^{a}

| | IC₅₀ (μM)^{b} | | | |
|---|---|---|---|---|
| Compound | SIRT1 | SIRT2 | SIRT3 | HDAC8 |
| Nicotinamide | 520 | | | |
| Peptide 1a | 1.7 ± 0.4^{c} | 1.8 ± 0.3 | 67.3 ± 2.4 | +^{c} |
| Peptide 2 | 116.8 ± 12.0 | 11.4 ± 1.1 | 449.4 ± 18.4 | —^{d} |
| Peptide 3a | 0.9 ± 0.2 | 4.3 ± 0.3 | 4.5 ± 2.0 | — |
| Peptide 4 | 10.4 ± 0.3 | ND^{e} | ND | ND |
| N^{α}-Fmoc-N^{ε}- | 2,000 (IC₂₅) | ND | ND | ND |
| thioacetyl- | | | | |
| lysine | | | | |
| N^{α}-acetyl-N^{ε}- | No inhibition | ND | ND | ND |
| thioacetyl- | at 2 mM | | | |
| lysine | | | | |
| Nicotinamide | 520 | ND | ND | NA^{f} |

^{a}Substrate concentrations used in an inhibition assay: 0.5 mM β-NAD⁺, 0.3 mM peptide substrate.
^{b}Mean ± standard deviation of duplicate measurements.
^{c}Sensitive to HDAC8.
^{d}Resistant to HDAC8.
^{e}Not determined.
^{f}Not applicable.

In yet another further embodiment, the present invention relates to the expanded use of the inhibitor design strategy of the present invention to develop Compound I as a method of prevention or treatment of a condition or disorder mediated by the protein deacetylation reaction catalyzed by human class III protein deacetylases SIRT4 and SIRT7 if a bona fide protein deacetylase activity is found for these two enzymes.

One further embodiment of the present invention relates to the expanded use of the inhibitor design strategy of the present invention to develop Compound I as a method of modulating the protein deacetylation activity of human class III protein deacetylases SIRT4 and SIRT7 if a bona ride protein deacetylase activity is found for these two enzymes.

The present invention further relates to the expanded use of the inhibitor design strategy of the present invention to develop Compound I as a method of prevention or treatment of a condition or disorder mediated by the protein deacetylation reaction catalyzed by further members within the human class III protein deacetylase enzyme family that are to be discovered.

The present invention still further relates to the expanded use of the inhibitor design strategy of the present invention to develop Compound I as a method of modulating the protein deacetylation activity of further members within the human class III protein deacetylase enzyme family that are to be discovered.

Although the invention has been described in detail with particular reference to certain embodiments detailed herein, other embodiments can achieve the same results. Variations and modifications of the present invention will be obvious to those skilled in the art and the present invention is intended to cover in the appended claims all such modifications and equivalents.

What we claim is:

1. A method for transforming a peptide substrate into a selective peptide inhibitor comprising:
a) providing a peptide substrate containing N^{ε}-acetyl-lysine;
b) providing an L-N^{α}-Fmoc-N^{ε}-thioacetyl-lysine building block; and
c) reacting the building block to replace N^{ε}-acetyl-lysine in the peptide substrate with N^{ε}-thioacetyl-lysine from the building block, wherein the resulting peptide exhibits selective inhibition for enzyme activity.

2. The method of claim 1 wherein the L-N^{α}-Fmoc-N^{ε}-thioacetyl-lysine is prepared by the condensation of L-N^{a}-Fmoc-lysine with ethyl dithioacetate.

3. The method of claim 1 wherein the resulting peptide of step (c) is human p53 tumor suppressor protein C-terminal peptide.

4. The method of claim 1 wherein the resulting peptide of step (c) is human α-tubulin peptide.

5. The method of claim 1 wherein the resulting peptide of step (c) is human Acetyl-coenzyme A synthetase 2 peptide.

6. A method for producing inhibitors for human class III protein deacetylases comprising:
a) providing a peptide substrate containing N^{ε}-acetyl-lysine;
b) providing an L-N^{α}-Fmoc-N^{ε}-thioacetyl-lysine building block; and
c) reacting the building block to replace N^{ε}-acetyl-lysine in the peptide substrate with N^{ε}-thioacetyl-lysine from the building block, wherein the resulting peptide exhibits selective inhibition for class III protein deacetylase enzymes.

7. The method of claim 6 wherein the resulting peptide demonstrates selective inhibition for SIRT1 sirtuin.

8. The method of claim 6 wherein the resulting peptide demonstrates selective inhibition for SIRT2 sirtuin.

9. The method of claim 6 wherein the resulting peptide demonstrates selective inhibition for SIRT3 sirtuin.

10. A peptide-based human sirtuin inhibitor comprising an NE-thioacetyl-lysine-containing peptide-based human sirtuin exhibiting resistance to classical HDAC-enzyme dethioacetylation.

11. The peptide of claim 10 wherein the peptide includes Compound I having the general formula:
R¹NH-φₘ-(ThAcK)-φₙ-COR² (I)
wherein, ThAcK is L-N^{ε}-thioacetyl-lysine; φ is one of the 20 naturally occurring L-amino acids and their D-counterparts (i.e. Alanine, Arginine, Asparagine, Aspartic acid, Cysteine, Glutamic acid, Glutamine, Glycine, Histidine, Isoleucine, Leucine, Lysine, Methionine, Phenylalanine, Proline, Serine, Threonine, Tryptophan, Tyrosine, Valine); m is 0-10; n is 0-10, and when m and/or n is >1, φ may be the same amino acid or different amino acids; R¹ is hydrogen (H), acetyl (CH₃CO), tert-butyloxycarbonyl (tBoc); R² is hydroxyl (OH), amino (NH₂).

12. The peptide of claim 12 wherein the peptide exhibits a sequence selected from the group consisting of H₂N-KKGQSTSRHK(ThAcK)LMFKTEG-COOH; H₂N-KKGQSTSRHK(AcK)LMFKTEG-COOH; H₂N-KKGQSTSRHK(K)LM FKTEG-COOH; H₂N-MPSD(ThAcK)TIGG-COOH; H₂N-KRLPKTRSG(ThAcK)VMRRLLRKII-COOH; H₂N-KRLPKTRSG(AcK)VMRRLLRKII-COOH; H₂N-KRLPKTRSG(K)VMRRLLRKII-COOH; and H₂N-HK(ThAcK)LM-COOH.

12. The peptide of claim 10 wherein the formation of the peptide includes the replacement of N^{ε}-acetyl-lysine with N^{ε}-thioacetyl-lysine.

13. The peptide of claim 11 wherein the formation of the peptide includes the replacement of N^{ε}-acetyl-lysine with N^{ε}-thioacetyl-lysine.
